(19) United States
(12) Patent Application Publication
(10) Pub. No.: US 2006/0030519 A1
(43) Pub. Date: Feb. 9, 2006

(54) Composition for reducing blood lipids

(75) Inventors: Frank Chiahung Mao, Taichung City (TW); Fan-Chin Hsiao, Taichung City (TW); Yi-Chung Chiang, Taipei City (TW)

Correspondence Address: BACON & THOMAS, PLLC, 625 SLATERS LANE, FOURTH FLOOR, ALEXANDRIA, VA, 22314, US

(73) Assignee: Maxluck Biotechnology Corp., Taipei City (TW)

(21) Appl. No.: 11/137,498

(22) Filed: May 26, 2005

(30) Foreign Application Priority Data
Aug. 5, 2004 (TW) 093123462

(51) Int. Cl. A61K 38/40; A61K 31/555

(52) U.S. Cl. 514/6; 514/184

(57) ABSTRACT

A composition for reducing the blood lipids is disclosed. The composition includes the lactoferrin and a trivalent chromium compound. The trivalent chromium compound of the present invention is selected from a group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof. The present invention also discloses a method for reducing the blood lipids of an acceptor.

### BACKGROUND OF THE INVENTION

1. Field of the Invention

The present invention relates to a composition and method for reducing the blood lipids and, more particularly, to a trivalent chromium dairy product that can reduce the blood lipids of an acceptor and the manufacturing method thereof.

2. Description of the Related Art

Owing to the development in economics, the change in lifestyle, and the abundance of foods, obesity is gradually found in all age groups of modern humans, from the children, the youth, to the aged people. The population of obese people keeps increasing and, accordingly, there are more and more people suffered from the derivative sickness of obesity, such as hypertension, heart disease, and hyperlipidemia. Therefore, it is really an important subject for the modem humans to study how to reduce the level of blood lipids.

Normally, the trivalent chromium absorbed from foods can be transferred into glucose tolerance factor (GTF) and then distributed in the tissues of human bodies. GTF in the tissues assistants blood lipids and hydrocarbons in undergoing normal metabolism through the synergistic effect with the insulin.

It is revealed from the research that the concentration of serum chromium decrease as one gets older. From the clinical researches in 1997, Davies verified that the concentration of serum chromium decreases from 0.5 ng/ml at one's childhood to 0.3 ng/ml at the age of 70 years old.

Obesity is a cause that drains chromium from a human body. Moreover, the deficiency of chromium will lead to problems in metabolism of blood lipids, which subsequently causes hyperlipidemia and other clinical symptoms.

Chromium may be assimilated in the forms of inorganic salt or organic salt from the daily food. However, the assimilation rate of inorganic chromium for human body is very low, and only ranges from 0.4% to 3%. The root cause lies in that the inorganic chromium tends to undergo olation reaction in the digestive tract. The olation reaction may produce bulky complex compounds that hinder the intestine tract from assimilating.

The adequate organic chromium includes chromium picolinate, chromium nicotinate, chromium GTF (Glucose Tolerance Factor), and chromium yeast extract.

The supplement of organic chromium helps to remedy the hyperlipidemia caused by the shortage of chromium. For the general adults, chromium combined with other kinds of vitamin and mineral substance may be deemed as a supplement of personal nutriment.

U.S. Pat. No. 4,923,855 disclosed a synthetic GTF chromium material and process therefore, in which the trivalent chromium is combined with nicotinic acid to obtain a novel chromium product having glucose tolerance factor. In 2002, Cefalu et al. announced that chromium picolinate could reduce the blood lipids of a obesity mouse.

### SUMMARY OF THE INVENTION

The present invention provides a composition for reducing blood lipids. More particularly, the present invention provides a composition of trivalent chromium compound and lactoferrin that can reduce the blood lipids. The present invention also provides a method for reducing the blood lipids of an acceptor. The method administrates an effective amount of a composition that reduces blood lipids to the acceptor. The composition is composed of trivalent chromium compound and lactoferrin.

The composition for reducing blood lipids of the present invention mainly includes (a) a lactoferrin and (b) a trivalent chromium compound.

The lactoferrin of the present invention is not restricted, and can come from cowmilk ferritin, goat milk ferritin, unpurified cowmilk, and unpurified goat milk. Because lactoferrin mainly exists in the whey of the milk, the lactoferrin of the present invention can also be replaced with whey protein products or milk products.

The trivalent chromium compound of the present invention is not restricted, too. Preferably, it can be selected from a group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

The inorganic salt of trivalent chromium includes, for example, chromium (III) chloride and chromium (III) sulfate.

The organic salt of trivalent chromium includes, for example, chromium (III) acetate, chromium picolinate, chromium nicotinate, amino acid chelated chromium, chromium yeast extract, and chromium yeast.

More preferably, the trivalent chromium compound is chromium (III) chloride hexahydrate, chromium (III) chloride, chromium (III) acetate, chromium (III) sulfate, chromium picolinate, or chromium nicotinate.

Generally speaking, the molar ratio of lactoferrin to the trivalent chromium compound of the present invention is not particularly restricted. Preferably, the molar ratio of lactoferrin to the trivalent chromium compound ranges from 1:200 to 10:1. More preferably, the molar ratio of lactoferrin to the trivalent chromium compound ranges from 1:20 to 1:1.

The composition of the present invention can serve as an additive of a dairy product. The dairy product can be the fresh milk of mammals, long-lived milk, concentrated milk, cheese, or milk powder.

The composition containing trivalent chromium lactoferrin of the present invention can be assimilated and utilized effectively by the human body. Taking the dairy product having the composition of the present invention not only can replenish the organic chromium efficiently, but also can control the level of blood lipids of a patient suffered from hyperlipidemia.

The composition containing trivalent chromium lactoferrin of the present invention is formed by mixing the trivalent chromium compound with the lactoferrin, and can enhance the normal metabolism of fat, carbohydrates, and protein. The lactoferrin is a glycoprotein that is capable of combining with metal ions. Each lactoferrin molecule can be combined with two trivalent chromium ions.

The composition of the present invention can be used to form a medicine. Also, it can be added into a dairy product, and thereby to form a dairy product containing trivalent chromium compound and lactoferrin, i.e., to form a food or nutriment.

The composition of the present invention can be taken by a patient suffered from hyperlipidemia because the composition can supplement the trivalent chromium effectively and enhance the normal metabolism of fat, carbohydrates, and protein. In addition, the level of blood lipids can be reduced to comfort the sufferers of hyperlipidemia.

### DETAILED DESCRIPTION OF THE PREFERRED EMBODIMENTS

The composition of the present invention can be formed by mixing the powder of lactoferrin with the powder of trivalent chromium compound. Moreover, water can also be added into the mixture of lactoferrin and the trivalent chromium compound to form a mixed solution. The mixed solution can be heated properly so that the mixing can be done adequately. The heating temperature ranges around 37° C. to 95° C., and preferably ranges from 50° C. to 80° C. The well-mixed solution is then spray-dried to form the composition containing trivalent chromium lactoferrin of the present invention.

The raw material of trivalent chromium compound used in the present invention can be the form of inorganic slat or organic slat, such as chromium (III) chloride hexahydrate, chromium (III) chloride, chromium (III) acetate, chromium (III) sulfate, chromium picolinate, and chromium nicotinate.

Lactoferrin could come from the solution or dry powder of lactoferrin. Because lactoferrin mainly exists in the whey of the milk, the present invention can also use an unpurified whey protein product or a dairy product to replace lactoferrin.

The following detailed description are given by way of example and not intended to limit the invention solely to the embodiments described herein.

### EXAMPLE 1

Mix 5 g of lactoferrin powder with 0.5 g of chromium (III) chloride hexahydrate to form the composition containing trivalent chromium lactoferrin of the present invention.

### EXAMPLE 2

Mix 5 g of lactoferrin powder and 0.5 g of chromium (III) chloride hexahydrate with 1 liter of water to form a solution. The solution is well-mixed and then spray-dried to form the composition containing trivalent chromium lactoferrin of the present invention.

### EXAMPLE 3

Mix 5 g of lactoferrin powder and 0.5 g of chromium (III) chloride hexahydrate with 1 liter of water to form a solution. The solution is well-mixed, spray-dried, and then mixed with 10 kg of milk powder to form the dairy product containing trivalent chromium lactoferrin.

### EXAMPLE 4

Mix 100 g of whey protein and 0.5 g of chromium (III) chloride hexahydrate with 3 liter of water to form a solution. The solution is well-mixed and then spray-dried to form the composition containing trivalent chromium lactoferrin of the present invention.

### EXAMPLE 5

The procedure of Example 4 is repeated first, and then the product is mixed with 10 kg of milk powder to form the dairy product containing trivalent chromium lactoferrin.

### EXAMPLE 6

The procedure of Example 4 is repeated, except that the mixed solution id added into 90 kg of fresh milk to form the dairy product containing trivalent chromium lactoferrin.

### EXAMPLE 7

Mix 5 g of lactoferrin powder with 0.3 g of chromium (III) chloride to form the composition containing trivalent chromium lactoferrin of the present invention.

### EXAMPLE 8

Mix 6 g of lactoferrin powder with 0.5 g of chromium acetate to form the composition containing trivalent chromium lactoferrin of the present invention.

### EXAMPLE 9

Mix 5 g of lactoferrin powder and 0.35 g of chromium sulfate with 1 liter of water to form a solution. The solution is well-mixed and then spray-dried to form the composition containing trivalent chromium lactoferrin of the present invention.

### EXAMPLE 10

Mix 5 g of lactoferrin powder and 0.8 g of chromium picolinate with 1 liter of water to form a solution. The solution is heated and mixed, and then spray-dried to form the composition containing trivalent chromium lactoferrin of the present invention.

### EXAMPLE 11

Mix 5 g of lactoferrin powder and 0.8 g of chromium nicotinate with 1 liter of water to form a solution. The solution is heated and mixed, and then spray-dried to form the composition containing trivalent chromium lactoferrin of the present invention.

### TEST EXAMPLE 1

The dairy product obtained from Example 5 is added into the feed of mice (Modified LabDiet w/35.5% Lard, PMI®) Richmond, Ind., USA). The experimental group includes three kinds of dairy products, each of which respectively has 200 ppb, 400 ppb, and 800 ppb of trivalent chromium. The control group has no trivalent-chromium dairy product. The experiment is carried out on male KK/HIJ mice with 10 weeks old. The concentration (mg/dl) of triglyceride in blood is measured before the experiment starts and at four weeks after the experiment starts. The result is listed in Table 1. The concentration of triglyceride in blood of mice fed with dairy product containing 800 ppb of trivalent chromium is obviously lower than that of mice fed without trivalent-chromium dairy product (P<0.05). Similarly, both the concentrations of triglyceride in bloods of experimental-group mice fed with dairy product containing 200 ppb and 400 ppb of trivalent chromium respectively are also lower than that of control group mice fed without trivalent-chromium dairy product.

TABLE 1
The variation of concentrations (mg/dl) of triglyceride in
bloods of male KK/H1J mice fed with dairy products containing
different kinds of concentration of trivalent chromium.

| | | | Four weeks after |
|---|---|---|---|
| | Number | Before the | the experiment |
| | of mice | experiment | starts |
| Contrast group | N = 12 | 198 ± 28 | 173 ± 35 |
| Fed with dairy product | N = 12 | 200 ± 26 | 152 ± 33 |
| containing 200 ppb of | | | |
| trivalent chromium | | | |
| Fed with dairy product | N = 12 | 207 ± 41 | 147 ± 33 |
| containing 400 ppb of | | | |
| trivalent chromium | | | |
| Fed with dairy product | N = 12 | 195 ± 33 | 141 ± 23 |
| containing 800 ppb of | | | |
| trivalent chromium | | | |

The composition containing trivalent chromium lactoferrins of the present invention can be taken by a patient suffered from hyperlipidemia because it can regulate the level of blood lipid thereof effectively. From Table 1, it is proven that the level of blood lipid is lowered effectively after the dairy product containing the composition of the present invention is taken.

Although the present invention has been explained in relation to its preferred embodiments, it is to be understood that many other possible modifications and variations can be made without departing from the scope of the invention as hereinafter claimed.

What is claims is:

1. A composition for reducing the blood lipids, comprising:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

2. The composition as claimed in claim 1, wherein the molar ratio of the lactoferrin to the trivalent chromium compound ranges from 1:200 to 10:1.

3. The composition as claimed in claim 1, wherein the molar ratio of the lactoferrin to the trivalent chromium compound ranges from 1:20 to 1:1.

4. The composition as claimed in claim 1, wherein the lactoferrin comes from the unpurified milk or the whey protein.

5. The composition as claimed in claim 1, wherein the lactoferrin comes from the group consisting of cowmilk ferritin, goat milk ferritin, unpurified cowmilk, unpurified goat milk, and combinations thereof.

6. The composition as claimed in claim 1, wherein the trivalent chromium compound is selected from the group consisting of chromium (III) chloride hexahydrate, chromium (III) chloride, chromium (III) acetate, chromium (III) sulfate, chromium picolinate, chromium nicotinate, and combinations thereof.

7. The composition as claimed in claim 1, wherein the composition serves as an additive of a dairy product, which is selected from a group consisting of fresh milk of mammals, long-lived milk, concentrated milk, cheese, and milk powder.

8. A method for reducing the blood lipids of an acceptor, comprising administrating an effective amount of a composition for reducing blood lipids to the acceptor, wherein the composition comprises:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

9. The method as claimed in claim 8, wherein the molar ratio of the lactoferrin to the trivalent chromium compound ranges from 1:200 to 10:1.

10. The method as claimed in claim 8, wherein the molar ratio of the lactoferrin to the trivalent chromium compound ranges from 1:20 to 1:1.

11. The method as claimed in claim 8, wherein the lactoferrin comes from the unpurified milk or the whey protein.

12. The method as claimed in claim 8, wherein the lactoferrin comes from a group consisting of cowmilk ferritin, goat milk ferritin, unpurified cowmilk, and unpurified goat milk.

13. The method as claimed in claim 8, wherein the trivalent chromium compound is selected from a group consisting of chromium (III) chloride hexahydrate, chromium (III) chloride, chromium (III) acetate, chromium (III) sulfate, chromium picolinate, and chromium nicotinate.

14. The method as claimed in claim 8, wherein the composition serves as an additive of a dairy product, which is selected from a group consisting of fresh milk of mammals, long-lived milk, concentrated milk, cheese, and milk powder.
